(19) United States
(12) Patent Application Publication
(10) Pub. No.: US 2026/0286263 A1
(43) Pub. Date: Sep. 24, 2026

(54) Organ-on-a-chip System and Organ-on-a-chip Package

(71) Applicant: DARWIN PRECISIONS CORPORATION, Hsinchu County (TW)

(72) Inventors: Ching-Fu Yang, Hsinchu County (TW); Pang-Chun Liu, Hsinchu County (TW)

(21) Appl. No.: 19/570,222

(22) Filed: Mar. 18, 2026

(30) Foreign Application Priority Data
Mar. 20, 2025 (TW) 114110592

(51) Int. Cl. C12M 3/00; C12M 1/00; C12M 1/34

(52) U.S. Cl. CPC C12M 21/08; C12M 23/02; C12M 23/34; C12M 23/38; C12M 23/40; C12M 23/48; C12M 41/44

(57) ABSTRACT

An organ-on-a-chip system is provided with a box structure, an organ-on-a-chip, a cover structure, and at least one tube. The box structure has an opening and includes at least one partition wall, and the interior of the box structure is divided into a plurality of spaces by the at least one partition wall. The organ-on-a-chip is disposed inside the box structure, and the organ-on-a-chip has at least one connecting hole connected to the at least one tube. The cover structure is disposed on the box structure and covers the interior of the box structure. The cover structure has at least one through hole corresponding to the at least one connecting hole of the organ-on-a-chip. The at least one tube is connected to the at least one connecting hole and the at least one through hole, and is adapted to allow fluid to pass through.

### FIELD OF THE INVENTION

The present invention relates to a field of bionic organs, and more particularly to an organ-on-a-chip and the use thereof.

### BACKGROUND OF THE INVENTION

An organ-on-a-chip (OOC) serves as an in vitro platform that can be used for culturing organoids and constructing physiological or pathological models for research and pharmaceutical development. Commercially available OOCs are typically housed within a packaging box and further sealed in a sterile bag. During use, the OOCs are removed from the sterile bag and packaging box, which are then discarded. The use of OOCs generally involves the supply of fluids, such as drug solution, gas exchange, and removal of metabolic waste. Accordingly, external fluidic tubes are typically connected to the OOCs to facilitate these functions during use.

### SUMMARY OF THE INVENTION

The present invention provides an organ-on-a-chip system to simplify the operation of the organ-on-a-chip.

The present invention further provides an organ-on-a-chip package that offers buffering and shock-absorbing effects and is also usable for the operation of the organ-on-a-chip.

In order to achieve one, some, or all of the aforementioned objectives, or other objectives, an organ-on-a-chip system is provided with a box structure, an organ-on-a-chip, a cover structure, and at least one tube. The box structure includes an opening and at least one partition wall. An interior of the box structure is divided into a plurality of spaces by the at least one partition wall. The organ-on-a-chip is adapted to be disposed within the box structure, and the organ-on-a-chip includes at least one connecting hole. The cover structure corresponds to the opening of the box structure and is adapted to be disposed on the box structure and cover the interior of the box structure. The cover structure includes at least one through hole corresponding to the at least one connecting hole of the organ-on-a-chip. The at least one tube is connected to the at least one connecting hole and the at least one through hole. The at least one tube is adapted to allow a fluid to pass through.

According to an embodiment of the present invention, an organ-on-a-chip package is further provided. The organ-on-a-chip package includes a packaging structure and an organ-on-a-chip. The packaging structure includes a box structure and a cover structure. The box structure is formed of a buffer material and includes an opening. The buffer material forms a support structure within the box structure. The cover structure corresponds to the opening of the box structure. The cover structure is disposed on the box structure and covers an interior of the box structure. The organ-on-a-chip is disposed within the packaging structure and carried on the support structure.

The present invention utilizes the box structure and the cover structure. In addition, the organ-on-a-chip is disposed within the interior of the box structure, and the cover structure is provided with the through hole. The box structure is divided into a plurality of spaces. The through hole of the cover structure corresponds to the connecting hole of the organ-on-a-chip. Thus, the operation process can be simplified by placing the cover structure on the box structure. In addition, the box structure is formed of a buffer material, and the buffer material also forms a support structure in the box structure, thereby serving as a packaging structure and providing support and shock-absorption for the organ-on-a-chip.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic exploded view of an organ-on-a-chip system according to an embodiment of the present invention.

FIG. 2 is a schematic diagram showing an assembly of the organ-on-a-chip system in FIG. 1.

FIG. 3 is a schematic perspective view of an organ-on-a-chip system according to another embodiment of the present invention.

FIG. 4 is a schematic exploded view of an organ-on-a-chip package according to another embodiment of the present invention.

### DETAILED DESCRIPTION OF PREFERRED EMBODIMENTS

In the following description of embodiments according to the present invention, terms indicating orientation or positional relationships, such as “upper,” “lower,” etc., are described based on the orientation or positional relationships shown in the corresponding drawings. These terms are used solely for the convenience of describing the invention and are not intended to limit the invention or to imply that the referenced elements must have a particular orientation or be constructed in a particular orientation. Furthermore, the terms “first,” “second,” and the like, as used in this specification or the claims, are employed merely to designate names of elements or to distinguish between different embodiments or ranges and are not intended to limit the number of elements. Unless limited otherwise, the terms “connected” or “coupled” as used in this specification or the claims may refer to either a direct connection between two elements without the presence of other elements or an indirect connection through the presence of other elements.

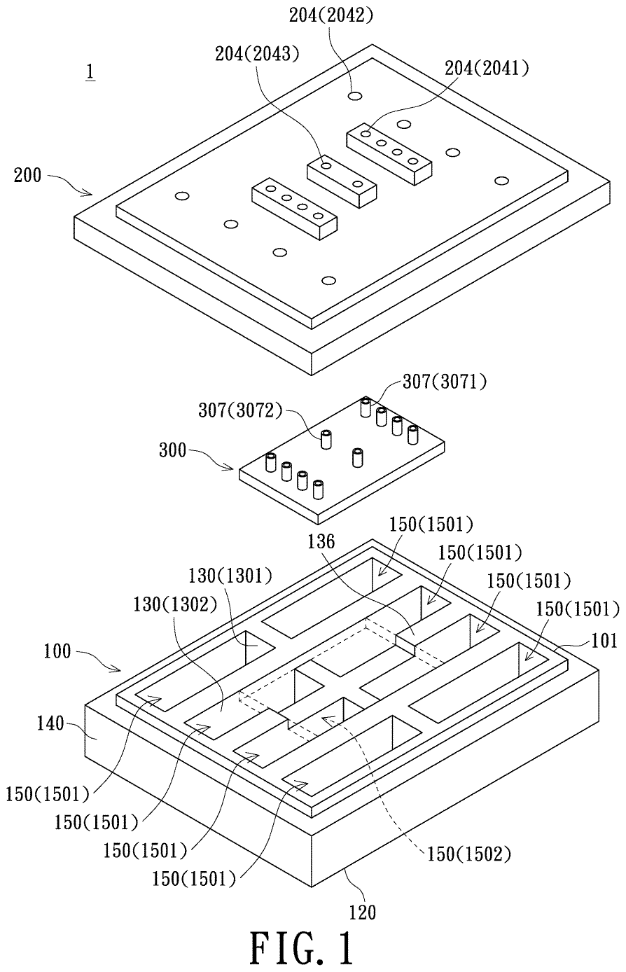

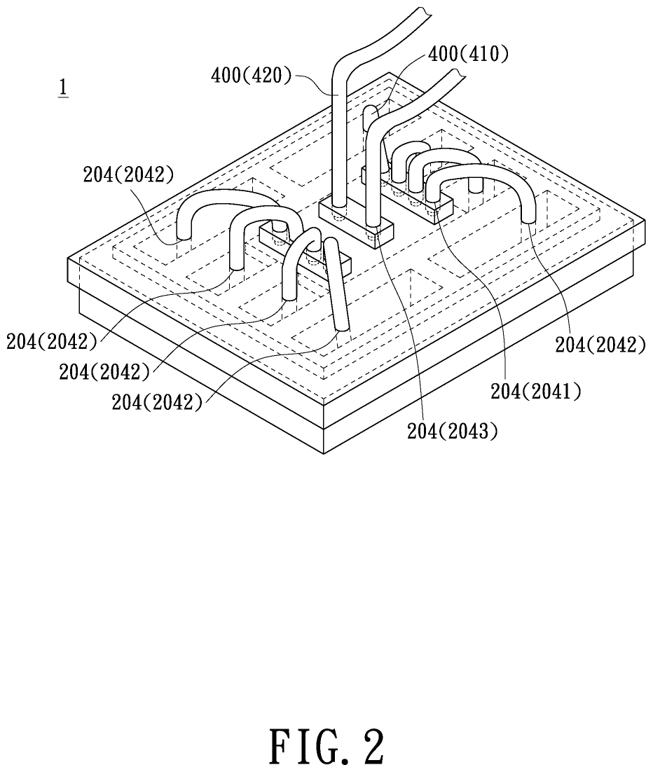

FIG. 1 is a schematic exploded view of an organ-on-a-chip system according to an embodiment of the present invention. FIG. 2 is a schematic diagram showing the assembly of the organ-on-a-chip system in FIG. 1. As shown in FIG. 1 and FIG. 2, according to an embodiment of the present invention, an organ-on-a-chip system 1 including a box structure 100, a cover structure 200, and an organ-on-a-chip 300 is provided. The organ-on-a-chip 300 is disposed within the box structure 100, and a quantity of the organ-on-a-chip 300 may be one or multiple. The cover structure 200 may be placed on and coupled to an opening 101 of the box structure 100, thereby covering an interior of the box structure 100. In FIG. 2, the box structure 100 and the organ-on-a-chip 300 covered by the cover structure 200 are shown in broken lines. An assembly of the box structure 100 and the cover structure 200 may be served as a packaging structure adapted for packaging the organ-on-a-chip 300.

As shown in FIG. 1, the box structure 100 includes at least one partition wall 130. A quantity of the partition wall 130 may be one or multiple, such as two, three, or more. The partition wall 130 divides the interior of the box structure 100 into a plurality of spaces 150. The plurality of spaces 150 are mainly separated by the partition wall 130. In other words, the spaces 150 may be enclosed by a portion of sidewalls 140 of the box structure 100, the partition wall 130, and a bottom 120 of the box structure 100. However, the invention is not limited thereto. In an embodiment of the present invention, the box structure 100 may be made of a plastic, and preferably a thermoplastic, for example, but is not limited to polyethylene terephthalate (PET), polystyrene (PS), polypropylene (PP), high-density polyethylene (HDPE), polyvinyl chloride (PVC), or polylactic acid (PLA). For example, the box structure 100 and the partition wall 130 are formed as a single unit by a sheet of thermoplastic material. In addition, the material of the box structure 100 is preferably a transparent or light-transmissive plastic. The cover structure 200 may be made of a plastic which is not limited to the same material of the box structure 100. The transparency or light-transmission of the box structure 100 and the cover structure 200 facilitates the observation of the organ-on-a-chip 300 in the organ-on-a-chip system 1.

A top portion of the partition wall 130 may be utilized as a supporting surface. For example, the organ-on-a-chip 300 is carried on the partition wall 130. The partition wall 130 may include at least one longitudinal partition wall 1301 and at least one transverse partition wall 1302. The organ-on-a-chip 300 may be carried on a plurality of parallel longitudinal partition walls 1301, a plurality of transverse partition walls 1302, or the longitudinal partition walls 1301 and the transverse partition walls 1302 which intersect each other. The top portion 136 of the partition wall 130 may be partially recessed toward the bottom 120 of the box structure 100 to form a retaining area for receiving the organ-on-a-chip 300. In other words, the partition wall 130 may form a space 150 in the box structure 100 away from the bottom 120 for receiving the organ-on-a-chip 300. A quantity of the retaining area or the space 150 (the space 150 that is away from the bottom 120) may depend on a quantity of the organ-on-a-chip 300, but is not limited thereto. For example, when the quantity of the organ-on-a-chip is two, the quantity of the retaining areas may also be two. In addition, the different organs-on-chips 300 may be received in the different retaining areas. The quantity of the retaining area or the space 150 may be different from the quantity of the organ-on-a-chip 300. For example, when the volume is sufficient, a single retaining area may receive a plurality of organs-on-chips 300.

The spaces 150 may further include a plurality of first receiving areas 1501 and at least one second receiving area 1502. The distinction between the first receiving areas 1501 and the second receiving area 1502 may lie in that the first receiving areas 1501 are positioned in a relatively lower portion within the box structure 100, that is closer to the bottom 120, whereas the second receiving area 1502 is positioned in a relatively higher portion within the box structure 100, that is closer to the cover structure 200. In an embodiment, the aforementioned spaces 150 enclosed by a part of the sidewall 140 of the box structure 100, the partition wall 130, and the bottom 120 of the box structure 100 may be regarded as the first receiving areas 1501. The retaining area formed in the top portion 136 of the partition wall 130 may be regarded as the second receiving area 1502. In an embodiment of the present invention, the first receiving areas 1501 may be receiving slots. Preferably, each of the receiving slots has a proper volume and is adapted to accommodate liquids. The second receiving area 1502 is adapted to accommodate the organ-on-a-chip 300.

The volume and/or the quantity of the first receiving areas 1501 may vary based on, for example, the size of the box structure 100, the configuration of the partition wall 130, the quantity of the spaces 150, and the organ-on-a-chip 300 (as described later). For example, as shown in FIG. 1, three transverse partition walls 1302 and the center longitudinal partition wall 1301 in the interior of the box structure 100 define the plurality of first receiving areas 1501 in the interior of the box structure 100. In the embodiment illustrated in FIG. 1, the quantity of the first receiving areas 1501 is eight, and the volume of each of the first receiving areas 1501 is substantially the same. However, the invention is not limited thereto. The quantity of the first receiving areas 1501 may be greater or fewer, and the volume of each of the first receiving areas 1501 may also differ from one another. For example, another box structure, having the same volume as the box structure 100, may have four of the first receiving areas, and the volume of each of the first receiving areas is larger than the volume of each of the first receiving areas 1501. In the present embodiment, each of the first receiving areas 1501 typically has a volume of 5 to 50 ml, such as 5 ml, 10 ml, 15 ml, 20 ml, 25 ml, 30 ml, 35 ml, 40 ml, 45 ml, and 50 ml. In fact, the volume is not limited and may be more than 50 ml. The volume of the first receiving areas 1501 may generally be adapted to correspond to the structure and the intended use of the organ-on-a-chip 300.

In some embodiments, the second receiving area 1502 may correspond to the organ-on-a-chip 300. Also, the size of the second receiving area 1502 may substantially match the organ-on-a-chip 300, thereby providing a positioning or retaining function. For example, the recessed portion in the center transverse partition wall 1302 has a length corresponding to a transverse length of the organ-on-a-chip 300, such that the organ-on-a-chip 300 abuts against the transverse partition wall 1302 in the transverse direction, thereby preventing lateral displacement. However, it is understood that the second receiving area 1502 is not limited to being formed by the recess in the top portion 136 of the partition wall 130.

To meet the requirements of research, pharmaceutical development, and other applications, the operation of the organ-on-a-chip 300 may include procedures such as loading of cells/tissues/organoids, drug administration, and observation of cells or tissues, for example, morphological observation and analysis of the culture medium. To perform such operations, the organ-on-a-chip 300 may be provided with an opening or a similar structure to allow communication between the interior and the exterior of the organ-on-a-chip 300. In the embodiment of the present invention, the organ-on-a-chip 300 further includes a connecting hole 307. Through the connecting hole 307, operations such as drug delivery and extraction of the culture medium can be performed on the cells or tissues within the organ-on-a-chip 300. The connecting hole 307 is adapted to connect the at least one tube for such operation. As shown in FIG. 1, the cover structure 200 includes a through hole 204, and the through hole 204 may correspond to the connecting hole 307 of the organ-on-a-chip 300. Here, the term “correspond to” may include, for example, positional correspondence or alignment between the connecting hole 307 and the through hole 204, as well as the compatibility for intended operations. In the embodiment, the cover structure 200 may further include a plurality of through holes 204, and at least one through hole 204 corresponds to the connecting hole 307. In addition, the organ-on-a-chip system 1 may include a plurality of organs-on-chips 300. At least some of the through holes 204 may correspond to the connecting holes 307 of the different organs-on-chips 300. It is understood that the cover structure 200 of the present invention is not limited to being provided only with the through hole 204 for the aforementioned operations. The cover structure 200 may have various functional designs corresponding to different types of the organs-on-chips 300.

As shown in FIG. 1, in the embodiment of the present invention, the organ-on-a-chip 300 may further include a plurality of connecting holes 307. The functions or structures of the connecting holes 307 may be the same or different. For example, the connecting holes 307 may have different structures and may be connected to different chambers of the organs-on-chips 300. However, the invention is not limited thereto. In an embodiment, the connecting holes 307 may be used for different functions such as drug delivery and extraction of the culture medium, though the invention is not limited thereto. Furthermore, the configuration of the connecting holes 307 is not limited to the illustrated embodiments.

The connecting holes 307 may further include a first connecting hole 3071, and the through holes 204 may further include a first through hole 2041. As shown in FIG. 2, when the organ-on-a-chip 300 is placed in the second receiving area 1502 within the box structure 100, and the cover structure 200 is placed on the box structure 100, the first through hole 2041 corresponds to and is aligned with the first connecting hole 3071. In addition, the first through hole 2041 and the first connecting hole 3071 may also be aligned and fitted to each other when the cover structure 200 is placed on the box structure 100. As shown in FIG. 2, in one embodiment, the organ-on-a-chip system 1 may further include at least one tube 400. The first through hole 2041 and the first connecting hole 3071 may be connected to the tube 400, and the tube 400 is adapted to allow the passage of fluids. The fluids may flow into or out of the organ-on-a-chip 300 through the tube 400, the first through hole 2041, and the first connecting hole 3071.

Here, the fluids may include gas, such as air, and liquids, such as the culture medium. The tube 400 may further include a first tube 410, and the first tube 410 may be connected to the first through hole 2041 and the first connecting hole 3071. In some embodiments, for example, the first tube 410 may be connected to the first through hole 2041 directly. When the cover structure 200 is coupled onto the box structure 100, and the first through hole 2041 is aligned with, corresponding to, and/or configured to the first connecting hole 3071, the first tube 410 may further be connected to the first connecting hole 3071. The connection between the first tube 410 and the first connecting hole 3071, as well as the fitting between the first through hole 2041 and the first connecting hole 3071, preferably provides airtightness, liquid-tightness, or a combination thereof. For example, an elastic sealing ring may be disposed at the connection or junction to achieve a tight fit. However, the invention is not limited thereto. In addition, the fitting between the first through hole 2041 and the first connecting hole 3071 may be achieved not only by coupling the cover structure 200 to the box structure 100, but also in combination with other operations.

As described above, the first receiving areas 1501 are adapted to accommodate liquids. The accommodated liquids may be the culture medium from the organ-on-a-chip 300 which includes the metabolic wastes of the cells, tissues, or organoids. In other words, the first receiving areas 1501 may be used to collect the culture medium or waste liquids. The liquids collected in the first receiving areas 1501 may subsequently be provided for detection and analysis. For example, by analyzing the composition of the waste liquids, biochemical and physiological information of the cells, tissues, or organoids can be obtained. In a preferred embodiment, the organ-on-a-chip 300 may deliver the culture medium or waste liquids to the first receiving areas 1501 of the box structure 100 through the first tube 410. Since the first receiving areas 1501 may be used to receive the liquids from the organ-on-a-chip 300, the configuration, quantity, or volume of the first receiving areas 1501 may vary according to, for example, the internal structure and the intended use of the organ-on-a-chip 300.

As shown in FIG. 1 and FIG. 2, the through holes 204 may further include a second through hole 2042, and preferably, the second through hole 2042 and the first through hole 2041 are arranged in pairs. That is, each of the first through holes 2041 may mate with a second through hole 2042. The first tube 410 may be connected to the second through hole 2042, and then connected to the first receiving area 1501 via the second through hole 2042. For example, the first tube 410 may be inserted from the exterior of the cover structure 200, pass through the second through hole 2042, and then enter the first receiving area 1501 within the box structure 100.

The arrangement of the second through hole 2042 on the cover structure 200 may correspond to the first receiving area 1501. For example, when the cover structure 200 is coupled to the box structure 100, a projection of the second through hole 2042 onto the box structure 100 may fall within the first receiving area 1501. In an embodiment of the present invention, the cover structure 200 may include a plurality of first through holes 2041 and a plurality of second through holes 2042. In addition, the quantity of the first through holes 2041 and the quantity of the second through holes 2042 may further correspond to a quantity of the first receiving areas 1501. For example, as illustrated in FIG. 1 and FIG. 2, the quantity of the first receiving areas 1501 is eight, and the quantity of the first through holes 2041 and the quantity of the second through holes 2042 may also be eight. The eight second through holes 2042 correspond to the first receiving areas 1501, respectively. However, the invention is not limited thereto. For example, the quantity of the first receiving areas 1501 may be different from the quantity of the first through holes 2041 and the quantity of the second through holes 2042. In the embodiment illustrated in FIG. 1 and FIG. 2, a quantity of the first tubes 410 is also eight, and the first tubes 410 are used to deliver the culture medium or waste liquids to the eight first receiving areas 1501, respectively. The invention is not limited thereto. In some embodiments, the same first receiving area 1501 may receive the culture medium or waste liquids through different first tubes 410.

In an embodiment of the present invention, the connecting holes 307 may further include a second connecting hole 3072, and the through holes 204 may further include a third through hole 2043. When the organ-on-a-chip 300 is disposed on the second receiving area 1502 of the box structure 100, and the cover structure 200 is mounted on the box structure 100, the third through hole 2043 may be aligned with and correspond to the second connecting hole 3072. In addition, the third through hole 2043 may be mated with the second connecting hole 3072 when the cover structure 200 is coupled to the box structure 100. The third through hole 2043 and the second connecting hole 3072 may be further connected to the tube 400.

The tubes 400 may further include a second tube 420, and the second tube 420 may be connected to the third through hole 2043 and the second connecting hole 3072. In some embodiments, for example, when the cover structure 200 is coupled to the box structure 100, and the third through hole 2043 and the second connecting hole 3072 are aligned with, engaged with, or correspond to each other, the second tube 420 may be further connected to the third through hole 2043 directly. The connection between the second tube 420 and the second connecting hole 3072 and the engagement between the third through hole 2043 and the second connecting hole 3072 may, as previously described, provide airtightness, liquid-tightness, or a combination thereof. The second tube 420 may extend from the cover structure 200, and may be further connected to components other than the organ-on-a-chip system 1. For example, the second tube 420 may be connected to a drug delivery device, allowing a drug to be introduced into the organ-on-a-chip 300 through the third through hole 2043 and the second connecting hole 3072. Alternatively, the second tube 420 may be connected to, for example, a gas cartridge to introduce gas into the organ-on-a-chip 300.

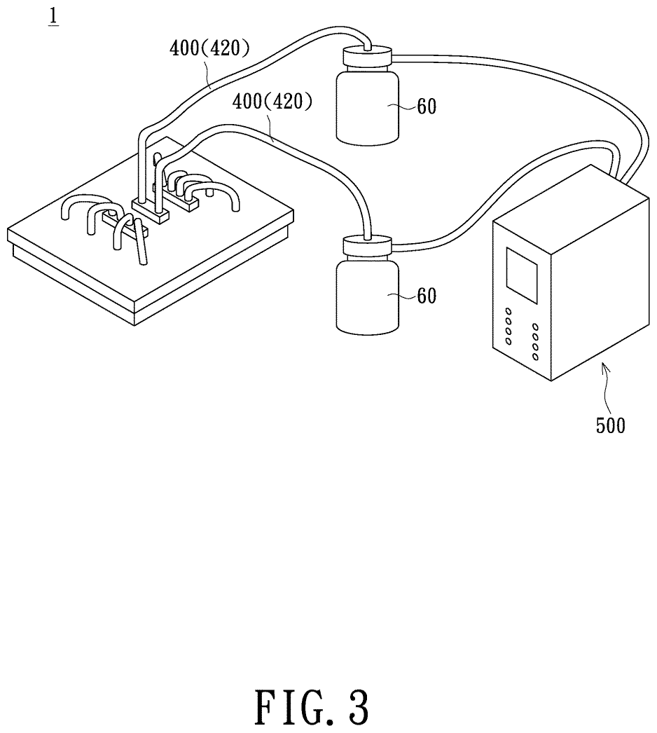

FIG. 3 is a schematic perspective view of an organ-on-a-chip system according to another embodiment of the present invention. In some embodiments, the organ-on-a-chip system 1 may further include a fluid control module 500. In an embodiment of the present invention, the tube 400 is adapted to be connected to the fluid control module 500. By using the fluid control module 500, fluids enter the organ-on-a-chip 300 through the tube 400, and may be discharged from the organ-on-a-chip 300 through the tube 400. For example, the fluid control module 500 may be connected to a drug delivery device for controlling a drug solution output, and/or may be connected to a gas cartridge for controlling a gas output. The fluid control module 500 may further drive liquids in the organ-on-a-chip 300 to flow out through the tube 400, for example, through the first tube 410. The discharged liquids may flow into the first receiving area 1501 through the tube 400, for example, through the second tube 420.

As shown in FIG. 3, the second tube 420 extends from the cover structure 200 and is connected to a drug delivery device 60. The drug delivery device 60 is connected to the fluid control module 500. By using the fluid control module 500, a drug solution may be delivered to the organ-on-a-chip 300 from the drug delivery device 60. The fluid control module 500 may be, for example, driven by electrical power. However, the invention is not limited thereto. The fluid control module 500 may be, for example, driven by non-electrical power. For example, the fluid control module 500 may be mechanically driven.

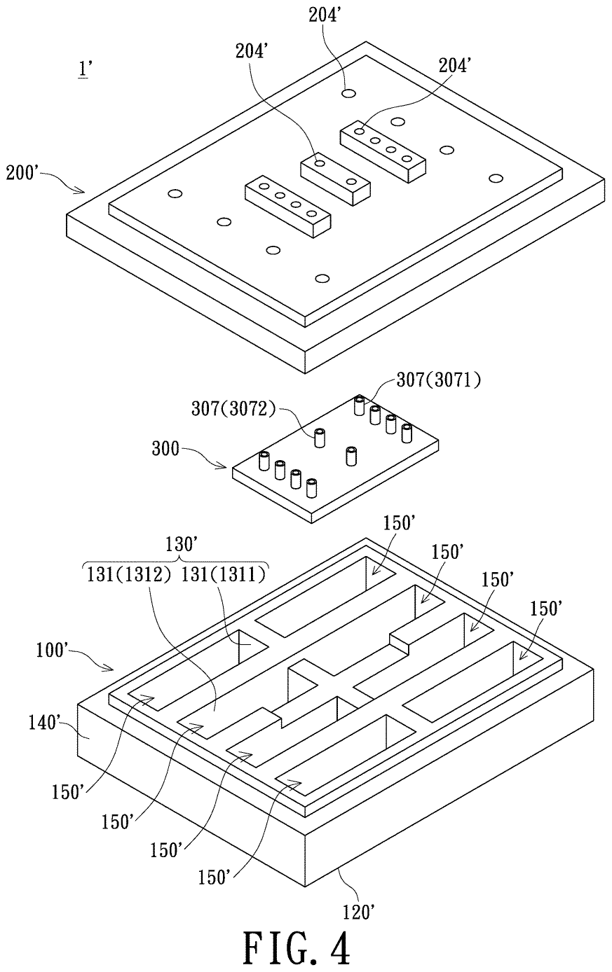

In an embodiment, the invention further provides a packaging structure for packaging the organ-on-a-chip. FIG. 4 is a schematic exploded view of an organ-on-a-chip package according to another embodiment of the present invention. As shown in FIG. 4, a packaging structure 10 including a box structure 100′ and a cover structure 200′ is provided. The organ-on-a-chip 300 is accommodated within the packaging structure 10. The combination of the organ-on-a-chip 300 and the packaging structure 10 is referred to as the organ-on-a-chip package 1′. There is no limitation on the number of the organs-on-chips 300 that may be accommodated within the packaging structure 10. That is, as shown in FIG. 4, the packaging structure 10 may accommodate a single organ-on-a-chip 300, or may accommodate multiple organs-on-chips 300.

The box structure 100′ of the packaging structure 10 may be formed of a buffer material. In addition, the buffer material may also form a support structure 130′ in the box structure 100′. In a preferred embodiment of the invention, the buffer material includes a plastic, a thermoplastic, a clamshell, or a combination thereof. The box structure 100′ and the support structure 130′ therein may be formed as a single unit.

The organ-on-a-chip 300 may be carried on the support structure 130′. The support structure 130′ may further include a retaining structure configured to accommodate the organ-on-a-chip 300 and to restrict movement thereof. In some embodiments, the support structure 130′ may have a configuration similar to the aforementioned partition wall 130 and may include a supporting wall 131. The supporting wall 131 may serve in a manner similar to the longitudinal partition wall 1301 and the transverse partition wall 1302 of the partition wall 130. The organ-on-a-chip 300 may be carried on the supporting walls 131. For example, the organ-on-a-chip is carried on a plurality of parallel longitudinal supporting walls 1311, on a plurality of parallel transverse supporting walls 1312, or on the longitudinal supporting walls 1311 and the transverse supporting walls 1312 that intersect with each other. The supporting walls 131 may divide the interior of the box structure 100′ into a plurality of spaces 150′ for receiving liquids, in a manner similar to the partition wall 130. Furthermore, the box structure 100′ may further have the same structure as the aforementioned box structure 100, and will not be repeated here.

In addition to being used for packaging the organ-on-a-chip 300, the packaging structure 10 may be used for the operation of the organ-on-a-chip 300. As described above, the term “operation” includes, for example, loading of cells, tissues, or organoids, drug administration, and observation of cells or tissues, such as observation of cell morphology and analysis of culture medium. The cover structure 200′ of the packaging structure 10 may further extend to include a structure adapted to assist with the operation of the organ-on-a-chip 300, for example, the through hole 204′ corresponding to the connecting hole 307 of the organ-on-a-chip 300. The cover structure 200′ may include the same structure as the cover structure 200 and will not be repeated here.

In summary, the box structure 100 and the cover structure 200 in the embodiment of the invention may not only be used to operate the organ-on-a-chip 300, but may also serve as the packaging structure 10 for the organ-on-a-chip 300. Since the cover structure 200 includes structures adapted to the organ-on-a-chip 300, including the through hole 204, and the through hole 204 may be adapted for connecting and guiding the tube 400, and since the box structure 100 includes the first receiving areas 1501 corresponding to the through hole 204, the tube 400, and the organ-on-a-chip 300, the connection between the tube 400 and the organ-on-a-chip 300 may be achieved by coupling the cover structure 200 to the box structure 100, thereby simplifying and standardizing the operation process. On the other hand, the box structure 100′ and the cover structure 200′ in the embodiment of the invention may not only be used to pack the organ-on-a-chip 300, but may also be used to operate the organ-on-a-chip 300. After the organ-on-a-chip 300 is taken from the organ-on-a-chip package 1′ according to the embodiment of the invention, the packaging structure 10 may be kept. After the cells, tissues, or organoids are loaded into the organ-on-a-chip 300, the packaging structure 10 may be used for subsequent operations. Since the step of connecting the tube 400 may be done by coupling the cover structure 200′ and the box structure 100′, the operation process may be further simplified. Moreover, since the spaces 150′ in the box structure 100′ may be adapted to collect the culture medium or waste liquids, the packaging structure 10 may be effectively utilized.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An organ-on-a-chip system, comprising:
a box structure, comprising an opening and at least one partition wall, wherein an interior of the box structure is divided into a plurality of spaces by the at least one partition wall;
an organ-on-a-chip, adapted to be disposed within the box structure, wherein the organ-on-a-chip comprises at least one connecting hole;
a cover structure, corresponding to the opening of the box structure and adapted to be disposed on the box structure and to cover the interior of the box structure, wherein the cover structure comprises at least one through hole corresponding to the at least one connecting hole of the organ-on-a-chip; and
at least one tube, connected to the at least one through hole and the at least one connecting hole, wherein the at least one tube is adapted to allow a fluid to pass through.

2. The organ-on-a-chip system according to claim 1, wherein the organ-on-a-chip is carried on the at least one partition wall.

3. The organ-on-a-chip system according to claim 2, wherein the box structure comprises a plurality of partition walls, the partition walls comprise at least one longitudinal partition wall and at least one transverse partition wall, the at least one longitudinal partition wall and the at least one transverse partition wall intersect with each other, and the organ-on-a-chip is carried on the at least one longitudinal partition wall and the at least one transverse partition wall.

4. The organ-on-a-chip system according to claim 1, wherein the spaces comprise a plurality of first receiving areas and at least one second receiving area, the at least one second receiving area is positioned closer to a top side of the cover structure than the first receiving areas, and the organ-on-a-chip is disposed in the at least one second receiving area.

5. The organ-on-a-chip system according to claim 4, wherein a top portion of the at least one partition wall is partially recessed to form the at least one second receiving area.

6. The organ-on-a-chip system according to claim 4, wherein the first receiving areas are a plurality of receiving slots, and each of the receiving slots is enclosed by sidewalls of the box structure, the at least one partition wall, or a combination thereof.

7. The organ-on-a-chip system according to claim 4, wherein the through holes comprise at least one first through hole and at least one second through hole, and the at least one first through hole and the at least one second through hole are arranged in pairs and provided in equal numbers.

8. The organ-on-a-chip system according to claim 7, wherein the at least one tube comprises at least one first tube, the at least one connecting hole comprises at least one first connecting hole, the at least one first tube is connected to the at least one first connecting hole, the at least one first through hole, and the at least one second through hole, and the at least one first tube is connected to one of the first receiving areas through the at least one second through hole.

9. The organ-on-a-chip system according to claim 8, wherein the at least one tube further comprises a second tube, the at least one connecting hole further comprises at least one second connecting hole, the through holes further comprise at least one third through hole, and the at least one second tube is connected to the at least one connecting hole and the at least one third through hole and extends outward from the cover structure.

10. The organ-on-a-chip system according to claim 9, wherein when the cover structure is arranged on the box structure, the at least one first through hole is aligned with the at least one first connecting hole, and the at least one third through hole is aligned with the at least one second connecting hole.

11. The organ-on-a-chip system according to claim 1, wherein the at least one tube is adapted to connect a fluid control module to allow the fluid to pass through.

12. An organ-on-a-chip package, comprising:
a packaging structure, comprising:
a box structure, formed of a buffer material and comprising an opening, wherein the buffer material forms a support structure within the box structure; and
a cover structure, corresponding to the opening of the box structure, wherein the cover structure is disposed on the box structure and covers an interior of the box structure; and
an organ-on-a-chip, disposed within the packaging structure, wherein the organ-on-a-chip is carried on the support structure.

13. The organ-on-a-chip package according to claim 12, wherein the buffer material comprises a thermoplastic.

14. The organ-on-a-chip package according to claim 13, wherein the box structure and the support structure are formed as a single unit by a sheet of the thermoplastic.

15. The organ-on-a-chip package according to claim 12, wherein the support structure comprises at least one support wall, and the organ-on-a-chip is carried on the at least one support wall.

16. The organ-on-a-chip package according to claim 15, wherein the support structure comprises a plurality of support walls, the support walls comprise at least one longitudinal support wall and at least one transverse support wall, the at least one longitudinal support wall and the at least one transverse support wall intersect each other, and the organ-on-a-chip is carried on the at least one longitudinal support wall and the at least one transverse support wall.

17. The organ-on-a-chip package according to claim 16, wherein the at least one longitudinal support wall and the at least one transverse support wall define a plurality of spaces within the box structure.

18. The organ-on-a-chip package according to claim 12, wherein the organ-on-a-chip further comprises at least one connecting hole adapted to connect a tube, the cover structure further comprises at least one through hole corresponding to the at least one connecting hole, and the at least one through hole is adapted to allow the tube to pass through.

19. The organ-on-a-chip package according to claim 12, wherein the packaging structure comprises a plastic, a thermoplastic, a clamshell, or a combination thereof.
